(19) United States
(12) Patent Application Publication
(10) Pub. No.: US 2004/0220669 A1
(43) Pub. Date: Nov. 4, 2004

(54) Intervertebral disk prosthesis

(76) Inventor: Armin Studer, Steinhausen (CH)

Correspondence Address: RANKIN, HILL, PORTER & CLARK, LLP, 925 EUCLID AVENUE, SUITE 700, CLEVELAND, OH, 44115-1405, US

(21) Appl. No.: 10/481,079

(22) PCT Filed: Jun. 27, 2001
(86) PCT No.: PCT/CH01/00403

(51) Int. Cl. A61F 2/44

(52) U.S. Cl. 623/17.12; 623/17.16

(57) ABSTRACT

An intervertebral disk prosthesis or a nucleus replacement prosthesis having an at least partially flexible jacket (3, 4, 5) that encloses a cavity (2) having a variable shape. The jacket (3, 4, 5) is at least partially configured as a semi-permeable membrane (3) for a solvent so that a solution of a substance contained in the cavity (2) can adapt its concentration and its volume to the external load conditions according to the principle of reverse osmosis.

The invention concerns an intervertebral disk prosthesis or a nucleus replacement prosthesis according to the generic part of patent claim 1.

Various intervertebral disk prostheses are already known from the state-of-the-art, including such which are capable to absorb water up to a certain extent, so that to achieve by virtue of this an increase of the volume. However, none of the known intervertebral disk prostheses are capable to reversibly adapt their volumes and shape within a certain limit as a result of external conditions, in particular external forces and loads acting on the prosthesis.

This is where the invention wants to provide remedy. The object of the invention is to produce an intervertebral disk prosthesis or a nucleus replacement prosthesis that is capable to assume in a reversible manner a greater or smaller, relative to the spinal column, height, depending from the loaded state (e.g. lying or standing patients).

The invention achieves this objective with an intervertebral disk prosthesis or a nucleus replacement prosthesis having the features of claim 1. By virtue of the semi-permeable membrane of the prosthesis according to the invention it is in the position in the implanted state to reversibly adapt its height, shape and elasticity as a function of external circumstances, i.e. whether the patient lies, stands or if there are additional loads on the spinal column (e.g. carrying loads or sporting activities). Because the prosthesis according to the invention is filled with a solution of a material, preferably of common salt, the concentration of which is higher than that of the body fluid, due to the osmotic effect it attempts to absorb water from the surrounding body fluid so that to dilute or equalise the concentration. Its volume increases on this occasion. As soon as the intervertebral disk prosthesis is subjected to a load, an effect opposing the osmotic effect occurs, according to which the water molecules are pressed outwards through the semi-permeable membrane until an equilibrium between the two effects takes place. At the same time the volume of the prosthesis is reduced and the concentration of the saline solution is increased again. The osmotic pressure is calculated from the formula TT=nRT/V, where n/V stands for the concentration of the solution in mol/L, R for the gas constant and T for the absolute temperature. Thus for an approx. 1% solution of common salt (corresponding to a concentration of approx. 0.3 mol/L) will result in a relatively high osmotic pressure of 7 to 8 bar.

Further advantageous developments of the invention are characterised in the dependent claims.

The advantages achieved by the invention are essentially that as a result of the prosthesis according to the invention the natural progress of absorption or emission of the water is carried out in the healthy intervertebral disk according to the same principle of osmosis. The dampening effect of the prosthesis also corresponds to that of the natural intervertebral disk. This dampening can be optimised by the concentration of the solution (saline solution) as well as by the thickness of the wall and/or the thickness of the jacket design. The traction forces, transferred from the active implant to the spinal column, help to tighten the anulus and consequently additionally introduce new rigidity into the moving segment of the spinal column.

In the case of a special embodiment the jacket comprises the semi-permeable membrane and has a casing, made from a biocompatible material, enclosing the membrane. This dense casing protects the body of the patient from the possible discharge of toxic materials which may occur during the life span of the implant of more than 20 years.

In the case of a further embodiment the casing is made from a polymer, preferably from polycarbonate-urethanes, silicone-polycarbonate-urethanes or silicone-polyetherurethanes. By virtue of their compatibility with the body these materials have proved themselves particularly suitable. To obtain a possibly desirable opaqueness for X-rays an X-ray contrasting agent like, for example barium sulphate, may be added.

The intervertebral disk prosthesis can be made from a homogeneous material or alternatively from two or several different materials.

In the case of a further embodiment the material of the casing, that encloses the semi-permeable membrane, can be welded together by an injection moulding process, wherein preferably a first partial casing obtained in a first injection extrusion moulding is completed by injection moulding with a second partial casing to produce a compact intervertebral disk prosthesis.

The casing has usefully a plurality of perforations, so that an outwardly open system is formed. The osmotic exchange can be additionally influenced and controlled by these perforations.

When using a sufficiently semi-permeable material for the casing, additional perforations in the casing may be omitted, so that the casing forms an outwardly closed system.

The intervertebral disk prosthesis can, for example, have a lens-shaped or balloon-shaped design. The advantage of these shapes is that in the collapsed shape the prosthesis has a small volume and after filling with a suitable solution it assumes a large volume. The filled balloon transmits the pressure on the implant uniformly in all directions and uniformly absorbs the pressure. In an advantageous manner the intervertebral disk prosthesis simulates a natural intervertebral disk.

The jacket of the intervertebral disk prosthesis may be a longitudinal receptacle with a coil, serpentine or spiral shape. The advantage of this design is the relatively small entry opening required to introduce this implant into the intervertebral space. Particularly advantageous is when the jacket has a material with memory effect of the shape.

The cavity of the intervertebral disk prosthesis is filled with a solution of a material in water (or in another solvent) either already prior to the implantation or subsequently through a suitable valve into the empty implanted semi-permeable receptacle. The dissolved material is preferably an inorganic salt (e.g. natrium chloride) or a sugar. The advantage, when using natrium chloride, is that it is physiologically harmless, so that no harmful materials would egress into the body even in the case of a leaky prosthesis.

The solution may also be a hydrogel. As hydrogels colloids, whose disperse phase (colloid) is combined with the continuous phase (water), are described, so that a viscous, gel-like product is the result. The osmosis effect is further optimised by controlling the swelling by means of the hydrogel.

The solution may also contain polymer or copolymer materials.

The concentration of the solution of a material should appropriately be at least 0.9%, preferably 2.0%. The molarity of the solution of a material should appropriately be at least 0.155 mol/L, preferably at least 0.3 mol/L. The molarity of the solution of a material should be maximum 3 mol/L, preferably maximum 5 mol/L.

The solution may additionally contain a contrasting agent for X-rays, preferably in liquid form, to make the implanted intervertebral disk prosthesis opaque for X-rays.

The semi-permeable membrane may be constructed from parchment paper, pig's bladder or a polymer, preferably a silicone. The semi-permeable membrane is preferably constructed as a virtually watertight pouch, that is filled with a solution of salt or sugar and is enveloped by a casing.

In the case of a special embodiment the minimum height of the intervertebral disk prosthesis is 4 mm, preferably 5 mm, the maximum height 15 mm, preferably 12 mm. The minimum volume of the cavity of the intervertebral disk prosthesis is 0.5 cm³, preferably 0.8 cm³, the maximum volume 5 cm³, preferably 4 cm³. The maximum height increase of the prosthesis due to water absorption is 8 mm, preferably 5 mm.

In the case of a further embodiment the jacket is fitted with a valve, so that the jacket can be implanted into the intervertebral space in the collapsed state by a laparoscope and then filled through the valve with an aqueous saline or sugar solution. The valve is appropriately provided on the periphery of the intervertebral disk prosthesis.

In the case of a further embodiment between the semi-permeable membrane enveloping the cavity and its casing a reservoir for pure solvent is provided.

The invention and developments of the invention are explained in detail based on the partly schematic illustrations of a number of embodiments.

They show in:

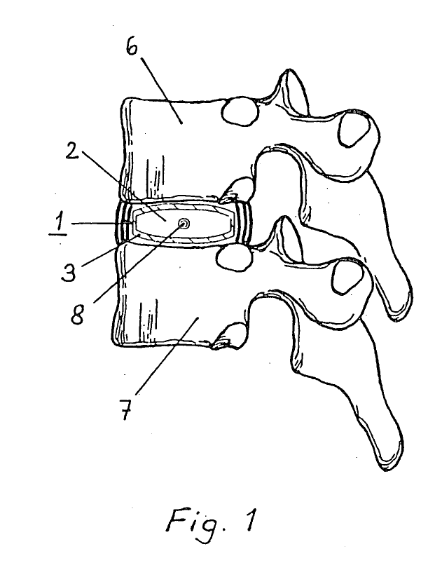

FIG. 1—a cross-section through two adjacent bodies of the vertebra with a prosthesis according to the invention having a balloon-shaped design;

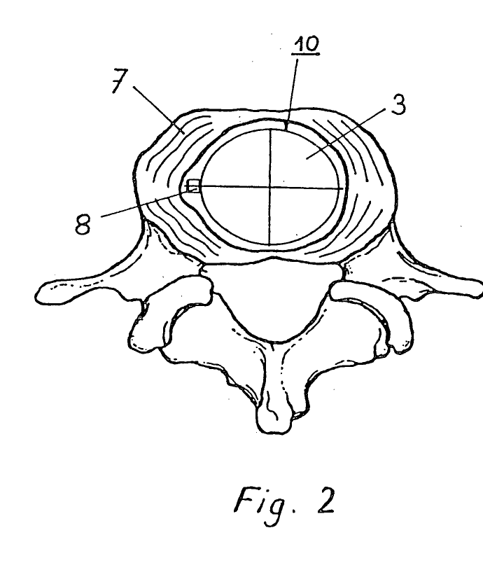

FIG. 2—a top view on a prosthesis according to the invention having a balloon-shaped design, that lies on the upper plate of a body of the vertebra;

FIG. 3—a top view on a prosthesis according to the invention having a coil-shaped design on the upper plate of a body of the vertebra;

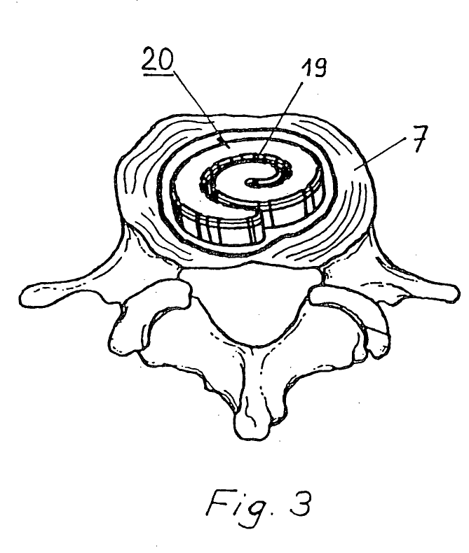

FIG. 4—a cross-section through a winding of the prosthesis according to FIG. 3 in the loaded state;

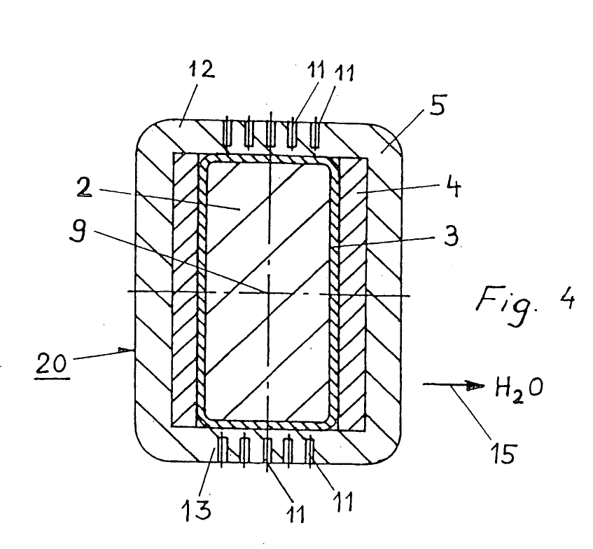

FIG. 5—a cross-section through a winding of the prosthesis according to FIG. 3 in the unloaded state, or in the swollen state;

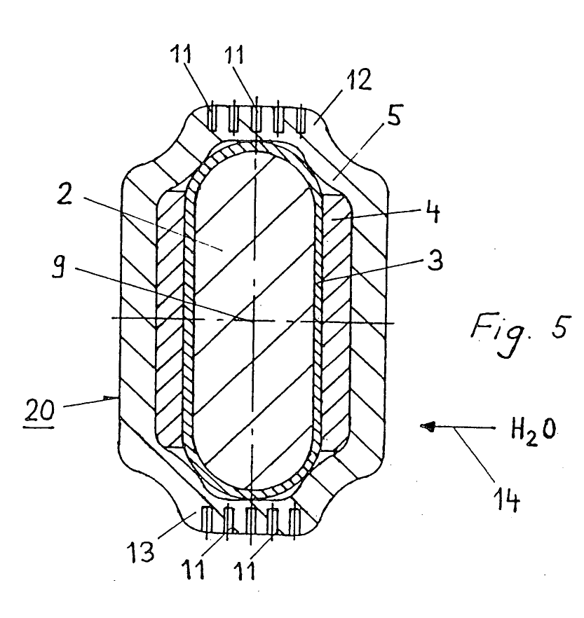

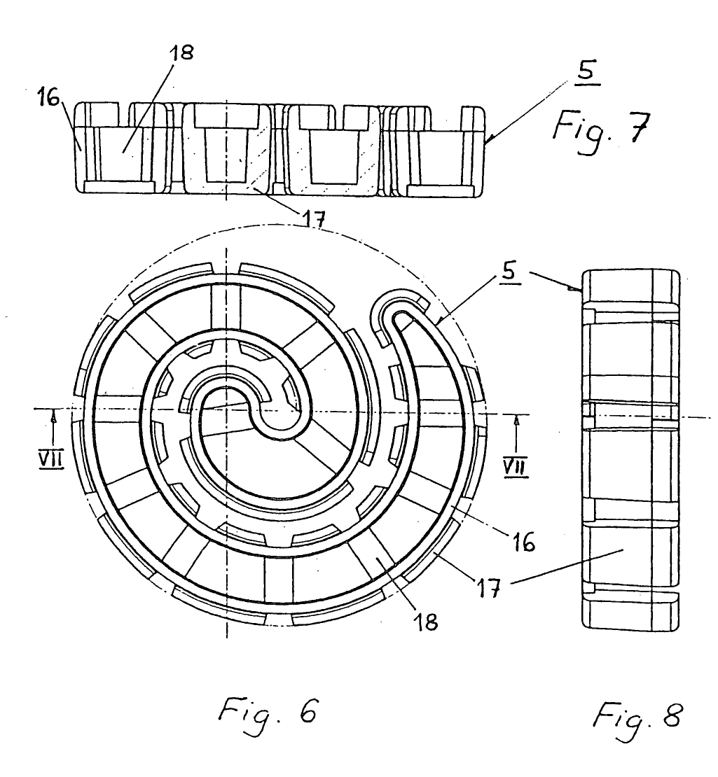

FIG. 6—a top view on the first partial casing of the prothesis according to FIGS. 4 and 5;

FIG. 7—a cross-section along line VII-VII of FIG. 6;

FIG. 8—a side view of the first partial shrouding according to FIG. 6;

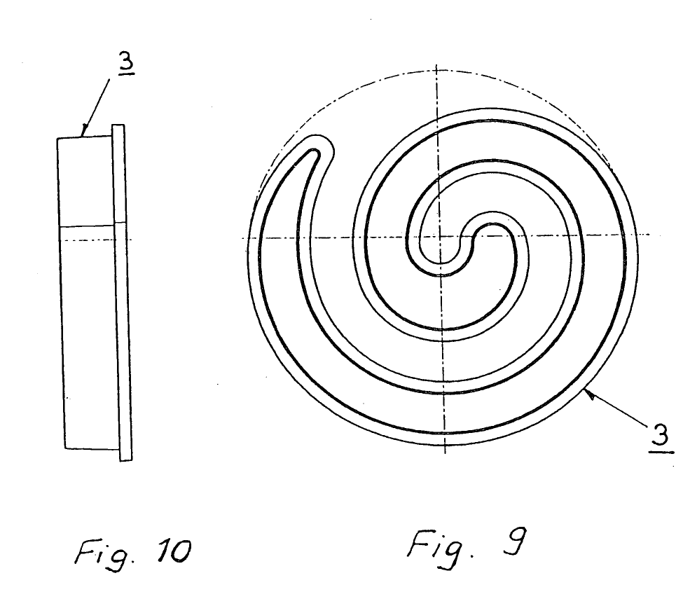

FIG. 9—a top view on the coil-shaped, semi-permeable membrane having a bellows shape of the prosthesis according to FIGS. 4 and 5;

FIG. 10—a side view of the semi-permeable membrane according to FIG. 9;

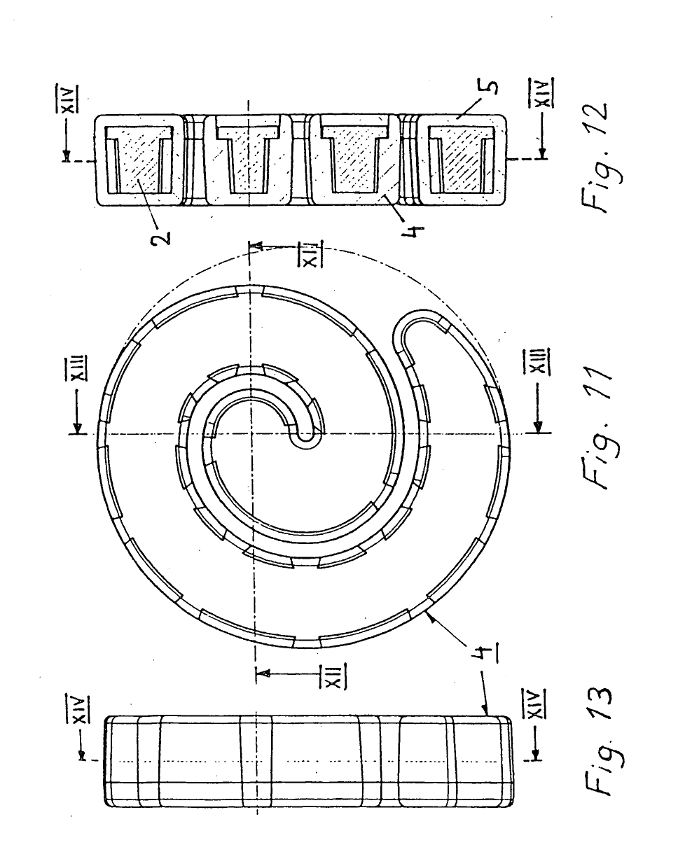

FIG. 11—a top view on the totally encased prosthesis according to FIGS. 4 and 5;

FIG. 12—a cross-section along the line XII-XII of FIG. 11;

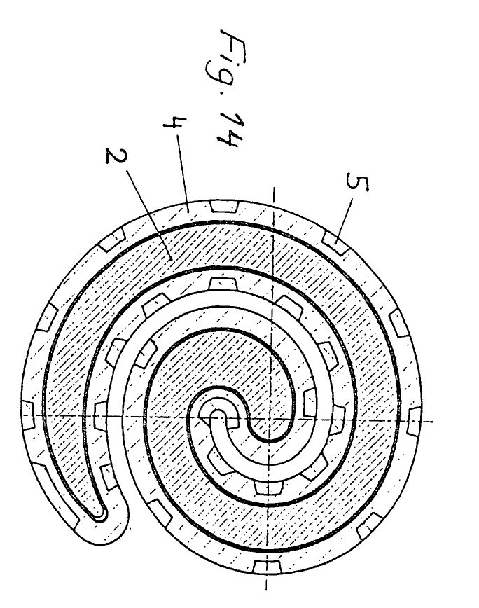

FIG. 13—a side view on the totally encased prosthesis according to FIG. 11 viewed in the direction of arrow XIII/XIII;

FIG. 14—a cross-section along the line XIV-XIV of FIG. 13.

FIG. 1 shows a nucleus replacement prosthesis 1 according to the invention in its simplest execution, wherein the jacket 3 is composed entirely from a semi-permeable membrane that, as a lens-shaped bag encloses a hollow space 2 filled with an aqueous natrium chloride solution. The nucleus replacement prosthesis 1 is implanted in the place of the prior removed, damaged natural nucleus pulposus, between two adjacent bodies 6, 7 of the vertebra. The nucleus replacement prosthesis 1 has a closable valve 8, so that in the unfilled collapsed state the intervertebral disk prosthesis can be implanted through an appropriate cannula in a least-invasive manner and filled through the valve 8 afterwards, with the aqueous natrium chloride solution and consequently brought to its lens-shaped form.

Alternatively, however, the valve 8 may be omitted and the lens-shaped bag initially filled with an aqueous natrium chloride solution. In this case the nucleus replacement prosthesis 1 produced in this manner has to be brought into the intervertebral space in the filled state.

FIG. 2 shows a variation of a nucleus replacement prosthesis 10, that is composed of a lens-shaped bag made of a plurality of materials, a thick-walled synthetic material in the zones of contact with the end plates of the bodies of the vertebra and a thin-walled semi-permeable synthetic material for the lateral surfaces of the jacket.

FIG. 3 shows a nucleus replacement prosthesis 20 with a variation as far as the shape is concerned, that instead of the lens-shaped design has a coil-shaped one. At the same time, as this is illustrated in FIG. 3, the individual windings may have an intermediate space 19 or abut against one another.

As it was already addressed in the explanation regarding FIG. 1, this intervertebral disk prosthesis 20 may also have a valve provided at the external end of the coil, through which a natrium chloride solution can be filled after the implantation had been carried out.

The variations illustrated in FIGS. 1 to 3 of nucleus replacement prostheses 1, 10, 20 may have, in addition to the semi-permeable membrane 3, an external casing to protect the semi-permeable membrane 3. At the same time the casing may have a single layered or a multi-layered construction. Such an embodiment, encased with two layers (sandwich structure) will be described below based on FIGS. 4 and 5.

FIG. 4 shows a cross-section orthogonally to the axis 9 of the coil through a winding of such an encased coil-shaped nucleus replacement prosthesis 20 (FIG. 3). The height of the nucleus replacement prosthesis 20 can be 4-15 mm (typically 8 mm). The hollow space 2 filled with an aqueous natrium chloride solution is totally enclosed by the relatively thin semi-permeable membrane 3. The latter is enclosed by a first partial casing 5 and a second partial casing 4. Both casings 4, 5 are composed of a biocompatible, biostable material, for example a polymer, in particular from silicone, polyurethane, polycarbonate-urethane, silicone-polycarbonate-urethane or silicone-polyetherurethane. All polymer materials can also be mixed with barium sulphate to make them opaque for X-rays. On the top side 12 and the bottom side 13 of the nucleus replacement prosthesis 20, i.e. on those surfaces of the jacket which come into contact with the cover plates of the bodies 6, 7 of the vertebra, holes 11, having a diameter of 0.01 μm to 1.2 mm (preferably between 20 μm to 0.2 mm), are provided essentially radially to the axis 9 of the coil. Their purpose is to more simply transport the body fluid, surrounding the nucleus replacement prosthesis 20, through the casings 4, 5 to the semi-permeable membrane 3, where the effect of the reversible osmosis can take place. The semi-permeable membrane 3 itself is not macro-perforated, but due to its structure it lets the small water molecules to pass through in both directions, i.e. into and out from the hollow space 2, whereas it is impermeable for the larger natrium and chloride ions. To further facilitate the transport of the body fluid to the semi-permeable membrane 3 lateral holes (not illustrated in the drawing) may also be provided in the casings 4, 5.

In the unloaded state, still or during the osmosis effect, the top side 12 and the bottom side 13 of the nucleus replacement prosthesis 20 illustrated in FIG. 5 exhibit a convex bulging relative to the axis 9 of the coil. As soon as intervertebral disk prosthesis 20 is subjected to a load, as the one arising in the implanted state for a standing patient, an effect, opposing the osmotic effect occurs, (absorption of water molecules from the body fluid with lower concentration into the saline solution of the hollow space 2 with higher concentration through the semi-permeable membrane 3, indicated by arrow 14), produced by the aqueous saline solution situated in the hollow space 2, meaning that the water molecules are pressed outward from the hollow space 2 through the semi-permeable membrane 3 (indicated by arrow 15) until an equilibrium occurs between the two effects (reversible osmosis). Due to the load on the convex top side 12 and bottom side 13 of the nucleus replacement prosthesis 20 illustrated, they will be partly or fully flattened as this is illustrated in FIG. 4.

Based on FIGS. 6-14 another possible construction of the nucleus replacement prosthesis 20 is explained.

In FIGS. 6-8 the first partial casing 5 is illustrated as an upwardly open coil. The coil-shaped double-wall 16 is bridged over by a plurality of U-shaped straps 17, so that a coil-shaped, interrupted absorption channel 18 will result for a bag made of a semi-permeable membrane 3, illustrated in FIGS. 9 and 10. This first partial casing 5 is produced in a first injection moulding die.

After placing the coil-shaped bag of a semi-permeable membrane 3, filled with an aqueous common saline solution and a liquid contrasting agent for X-ray, into the first partial casing 5, the thus prepared, intermediate component is placed into a further (second) injection moulding die, so that to close in this manner the still existing gaps and interruptions of the first partial casing 5 by means of a second injection moulding process. The second partial casing 4, produced in this manner, complements fully the partial casing 5, so that by virtue of this second injection moulding process the coil-shaped bag with the semi-permeable membrane 3, filled with the saline solution, is totally enclosed by the casings 4, 5 complementing one another, thus producing the intervertebral disk prosthesis 20, illustrated in FIG. 14.

Consequently, the saline solution filled into the hollow space 2 can, according to the principle of the reversible osmosis, adapts its concentration and its volume to suit the external loaded state of the implanted intervertebral disk prosthesis 20, enveloped by the body fluid.

1. An intervertebral disk prosthesis (1, 10, 20) or a nucleus replacement prosthesis with an at least partly flexible jacket (3, 4, 5) that encloses a cavity (2) with a varying shape, the jacket constructed at least partially as a semi-permeable membrane (3) for a solvent, wherein the jacket (3, 4, 5) is a longitudinal receptacle having a shape selected from the group consisting of spiral, serpentine and coil.

2. The intervertebral disk prosthesis according to claim 2, wherein the jacket (3, 4, 5) comprises the semi-permeable membrane (3) and has a casing (4, 5) made from a biocompatible material that encloses the membrane (3).

3. The intervertebral disk prosthesis according to claim 2, wherein the casing (4, 5) is made from a polymer.

4. The intervertebral disk prosthesis according to claim 2, wherein the casing (4, 5) is made from a homogeneous material.

5. The intervertebral disk prosthesis according to claim 2, wherein the casing (4, 5) is made from at least two different materials.

6. The intervertebral disk prosthesis according to claim 2, wherein the material of the casing (4, 5), that encloses the semi-permeable membrane (3), is welded together by an injection molding process, whereby a first partial casing (5) obtained by a first injection extrusion molding step is completed by injection molding with a second partial casing to produce a compact intervertebral disk prosthesis.

7. The intervertebral disk prosthesis according to claim 2, wherein the casing (4, 5) has perforations so that it forms an outwardly open system.

8. The intervertebral disk prosthesis according to claim 2, wherein the casing (4, 5) forms an outwardly closed system.

9. The intervertebral disk prosthesis according to claim 1, wherein the prothesis's shape is selected from the group consisting of lens-shaped and balloon-shaped.

10. The intervertebral disk prosthesis according to claim 1, wherein the jacket (3, 4, 5) is a coil-shaped longitudinal receptacle in which individual coil windings have an intermediate space (19).

11. The intervertebral disk prosthesis according to claim 1, wherein the jacket (3, 4, 5) is a material having a memory effect of the shape.

12. The intervertebral disk prosthesis according to claim 1, wherein the cavity (2) is filled with a solution of a material in water or in another solvent, while the dissolved material is selected from the group consisting of organic salts and sugar.

13. The intervertebral disk prosthesis according to claim 12, wherein the solution is a hydrogel.

14. The intervertebral disk prosthesis according to claim 12, wherein the solution contains polymer or copolymer materials.

15. The intervertebral disk prosthesis according to claim 12, wherein the inorganic salt is natrium chloride.

16. The intervertebral disk prosthesis according to claim 1, wherein the semi-permeable membrane (3) is made from a material selected from the group consisting of parchment paper, pig's bladder, and a polymer.

17. The intervertebral disk prosthesis according to claim 1, wherein said prosthesis has a laminate structure.

18. The intervertebral disk prosthesis according to claim 2, wherein the semi-permeable membrane (3) is constructed as a virtually watertight pouch that is filled with a solution of salt or sugar and is enveloped by a casing (4, 5).

19. The intervertebral disk prosthesis according to claim 1, wherein a height of the prosthesis is between 4-5 mm.

20. The intervertebral disk prosthesis according to claim 1, wherein a maximum height of the prosthesis is between 12-15 mm.

21. The intervertebral disk prosthesis according to claim 1, wherein a minimum volume of the cavity (2) is between 0.5-0.8 cm³.

22. The intervertebral disk prosthesis according to claim 1, wherein a maximum volume of the cavity (2) is between 4-5 cm³.

23. The intervertebral disk prosthesis according to claim 1, wherein a maximum height increase due to water absorption is between 5-8 mm.

24. The intervertebral disk prosthesis according to claim 1. wherein the jacket (3, 4, 5) is fitted with a valve so that the jacket (3, 4, 5) can be implanted into the intervertebral space in the collapsed state by a laparoscope and then filled through the valve with an aqueous saline or sugar solution.

25. The intervertebral disk prosthesis according to claim 24, wherein the valve is provided on a periphery of the intervertebral disk prosthesis.

26. The intervertebral disk prosthesis according to claim 2, wherein, between the semi-permeable membrane (3) enveloping the cavity (2) and the casing (4, 5), a reservoir for pure solvent is provided.

27. The intervertebral disk prosthesis according to claim 1, wherein the concentration of the solution of a material is between about 0.9% to 2.0%.

28. The intervertebral disk prosthesis according to claim 12, wherein a molarity of the solution of a material is between about 0.155-0.3 mol/L.

29. The intervertebral disk prosthesis according to claim 12, wherein a maximum molarity of the solution of a material is between about 3-53 mol/L.

30. The intervertebral disk prosthesis according to claim 12, wherein the solution additionally contains a contrasting agent for X-rays.

31. The intervertebral disk prosthesis according to claim 1, wherein said prosthesis' shape simulates a natural intervertebral disk.

32. The intervertebral disk prosthesis according to claim 1, wherein the casing (4, 5) contains a material that is opaque for X-rays.
